(19) United States
(12) Patent Application Publication
(10) Pub. No.: US 2025/0277800 A1
(43) Pub. Date: Sep. 4, 2025

(54) NUCLEIC ACID SEQUENCES RESPONSIVE TO CREATININE IN CLINICALLY USEFUL RANGES

(71) Applicant: THE TRUSTEES OF COLUMBIA UNIVERSITY IN THE CITY OF NEW YORK, New York, NY (US)

(72) Inventors: Milan STOJANOVIC, Ridgewood, NJ (US); Nenad MILOSAVIC, Hackensack, NJ (US); Sumit MOHAN, Irvington, NY (US); Jovan JOVICIC, Swarthmore, PA (US); Jacob STEVENS, New York, NY (US)

(73) Assignee: THE TRUSTEES OF COLUMBIA UNIVERSITY IN THE CITY OF NEW YORK, New York, NY (US)

(21) Appl. No.: 18/592,917

(22) Filed: Mar. 1, 2024

(51) Int. Cl. G01N 33/70; C12N 15/115

(52) U.S. Cl. CPC G01N 33/70; C12N 15/115; C12N 2310/531

(57) ABSTRACT

Aptamers and methods for detecting a target molecule/analyte in a sample are disclosed herein. The aptamer can include a single-stranded deoxyribonucleic acid (DNA) strand that includes an oligonucleotide sequence with bases identical at least about 60% to TAATTGTGGTTCGTGTAAA (SEQ ID NO: 1). The aptamer can be configured to bind the target molecule/analyte with a dissociation constant between about 10⁻⁹ and about 10⁻³ M.

### CROSS-REFERENCE TO RELATED APPLICATIONS

This application is a Continuation of International Patent Application No. PCT/US22/42518, filed on Sep. 2, 2022, which claims priority to U.S. Provisional Application Ser. No. 63/240,192, filed on Sep. 2, 2021, which are incorporated by reference herein in their entireties.

### GRANT INFORMATION

This invention was made with Government Support under Grant Nos. GM138843, EB022015, and DA045550 awarded by the National Institutes of Health. The Government has certain rights in the invention.

### SEQUENCE LISTING

This application contains a Sequence Listing, which has been submitted in XML format via EFS-Web and is hereby incorporated by reference in its entirety. Said XML copy, created on Feb. 29, 2024, is named 070050.6808.xml and is 38.334 bytes in size.

### BACKGROUND

Creatinine can be a waste product that comes from the metabolism of the muscles. As kidney disease progresses, the level of creatinine in a body fluid (e.g., serum, blood, urine, etc.) can rise, and creatinine levels in the blood or urine can be used to assess whether the kidneys are working properly. However, accurate measurement of creatinine can be challenging, as certain systems and techniques are not sensitive enough to detect a relatively low level of creatinine. Furthermore, the blood or urine can include certain molecules/analytes (e.g., magnesium, potassium, calcium, or sodium) that can hinder the accurate measurement of creatinine levels requiring further buffering solutions. Such buffering solutions can further decrease the accuracy of the creatinine measurement in clinically relevant ranges.

Thus, there remains a need for techniques for direct measurements of target molecules under clinical or physiological conditions without further buffering.

### SUMMARY

The disclosed subject matter provides aptamers and methods for detecting a target molecule/analyte in a sample.

An example aptamer for detecting a target molecule/analyte in a sample can include a single-stranded deoxyribonucleic acid (DNA) strand. In non-limiting embodiments, the single-stranded DNA strand can include an oligonucleotide sequence with bases identical to at least about 60% to TAATTGTGGTTCGTGTAAA (SEQ ID NO: 1). In non-limiting embodiments, the aptamer can be configured to bind the target molecule/analyte with a dissociation constant between about 10⁻⁹ and about 10⁻³ M.

In certain embodiments, the target molecule/analyte can be creatinine. In non-limiting embodiments, the sample can include blood, serum, effluent, saliva, sweat, tears, or combinations thereof.

In certain embodiments, the single-stranded DNA strand can include at least about fifteen bases.

In certain embodiments, the aptamer can include at least one modification. In non-limiting embodiments, the at least one modification can include atom substitution, neutralization of negative charges, introduction of positive charges, or combinations thereof. In non-limiting embodiments, the at least one modification can be a modified base. The modified base can include a ribonucleic acid (RNA), a modified RNA, a modified DNA, a peptide nucleic acid (PNA), or combinations thereof.

In certain embodiments, the aptamer can include a functional group. In non-limiting embodiments, the functional group can include thiols, phosphothiols, carboxyl, amines, carbonyls, aldehydes, alkynes, azides, alkenes, strained alkenes, tetrazines, and/or products thereof.

In certain embodiments, the aptamer can be a stem-loop aptamer that can include a capture region and a stem region. The stem region can be configured to be positioned to transform a second conformation into a stem-loop structure of the aptamer, or stem-loop structure into a second conformation when the oligonucleotide sequence binds to the target molecule/analyte.

In certain embodiments, the aptamer can be modified or configured to be immobilized to a substrate for sensing the target molecule/analyte. In non-limiting embodiments, the stem-loop structure can be modified to move away from the substrate upon binding to the target molecule/analyte. In non-limiting embodiments, the stem-loop structure can be modified to approach to the substrate upon binding to the target molecule/analyte.

In certain embodiments, the aptamer can be configured to be incorporated into a sensor device. In non-limiting embodiments, the sensor device can include a field-effect transistor and the aptamer. In non-limiting embodiments, the aptamer can be configured to be incorporated into a sensor device. The sensor device can include a gold substrate and an aptamer. In non-limiting embodiments, the aptamer can be configured to be incorporated into a sensor device. The sensor device can include a fiber-optical cable and an aptamer. In non-limiting embodiments, the aptamer can be configured to be incorporated into a sensor device. The sensor device can include a quartz surface and an aptamer.

In certain embodiments, the aptamer can be configured to the target molecule/analyte without being freely diffused. In non-limiting embodiments, the sensor device can include a freely diffusing molecular with a molecular weight between about 1,000 D and about 1,000,000 D.

In certain embodiments, the aptamer can be attached to a fluorophore, a quencher, an enzyme, a redox dye, or combinations thereof.

In certain embodiments, the sample can be a diluted sample. The diluted sample can be a sample diluted up to about 1%, 10%, 20%, 30%, 40%, 50%, 60%, 70%, 90%, 100%, 200%, 300%, 400%, 500%, 600%, 700%, 800%, 900%, or 1000% with a solution. In non-limiting embodiments, the sample can be a non-diluted sample.

The disclosed subject matter provides methods for detecting a target molecule/analyte in a sample. An example method can include contacting at least a portion of the sample with effective amounts of an aptamer and detecting a change after the contacting. In non-limiting embodiments, the aptamer can include a single-stranded deoxyribonucleic acid (DNA) strand that includes an oligonucleotide sequence with bases identical at least about 60% to TAATTGTGGTTCGTGTAAA (SEQ ID NO: 1). In non-limiting embodiments, the aptamer can be configured to bind the target molecule/analyte with a dissociation constant between about 10⁻⁹ and about 10⁻³ M.

In certain embodiments, the change can include a change of conductance, fluorescence, and/or any electrochemical readouts.

In certain embodiments, the target molecule/analyte can be creatinine. In non-limiting embodiments, the sample can include blood, plasma, serum, effluent, saliva, sweat, tears, or combinations thereof.

In certain embodiments, the aptamer can be a stem-loop aptamer that includes a capture region and a stem region. In non-limiting embodiments, the stem region can be configured to be positioned to transform a stem-loop structure of the aptamer to a second conformation when the oligonucleotide sequence binds to the target molecule/analyte.

In certain embodiments, the aptamer can be configured to be immobilized to a substrate for sensing the target molecule/analyte. In non-limiting embodiments, the substrate can include gold and/or quartz.

In certain embodiments, the stem-loop structure can be modified to move away from the substrate upon binding to the target molecule/analyte so the conductance of the substrate changes. In non-limiting embodiments, the stem-loop structure can be modified to approach to the substrate upon binding to the target molecule/analyte so the conductance of the substrate changes.

In certain embodiments, the aptamer can be attached to a fluorophore, a quencher, an enzyme, a redox dye, or combinations thereof.

In certain embodiments, the sample can be a diluted sample, wherein the sample is a diluted sample, wherein the diluted sample is a sample diluted up to about 1%, 10%, 20%, 30%, 40%, 50%, 60%, 70%, 90%, 100%, 200%, 300%, 400%, 500%, 600%, 700%, 800%, 900%, or 1000% with a solution. In non-limiting embodiments, the sample can be a non-diluted sample.

In certain embodiments, the method can further include diagnosing a kidney disease based on the detected level of creatinine in the sample and providing a subject with a treatment based on the detected level of creatinine in the sample.

In certain embodiments, a level of the treatment can be enhanced with an increased creatinine level detected by the aptamer. In non-limiting embodiments, a level of the treatment can be lowered with a decreased creatinine level detected by the aptamer.

In certain embodiments, the method can further include sensing the target molecule/analyte at a predetermined frequency. In non-limiting embodiments, the predetermined frequency can be once every hour, once every ten minutes, once every minute, once every second, or any predetermined period in between.

### BRIEF DESCRIPTION OF THE DRAWINGS

Further features and advantages of the present disclosure will become apparent from the following detailed description taken in conjunction with the accompanying figures showing illustrative embodiments of the present disclosure, in which:

FIG. 1 (SEQ ID NOS: 2-5) shows example sequences that can be identified within isolated sequences in accordance with the present disclosure.

FIG. 2 (SEQ ID NOS: 6-9) shows example sequences that can be identified within isolated sequences in accordance with the present disclosure.

FIG. 3 (SEQ ID NO: 10) shows example sequences that can be attached to solid surfaces in accordance with the present disclosure.

FIG. 4 (SEQ ID NOS: 11-12) shows example sequences that can be used to sense creatinine in a solution in accordance with the present disclosure.

FIG. 5 shows example response ranges of the disclosed sequences to creatinine in accordance with the present disclosure.

FIG. 6 provides graphs showing raw monitoring and ON-OR monitoring of the disclosed sequences using Square Wave Voltammetry in accordance with the present disclosure.

Throughout the figures, unless otherwise stated, are used to denote like features, elements, components, or portions of the illustrated embodiments. Moreover, while the present disclosure will now be described in detail with reference to the figures, it is done so in connection with the illustrative embodiments.

### DETAILED DESCRIPTION

The disclosed subject matter provides techniques for detecting a target analyte. The disclosed techniques can utilize nucleic acids that are responsive to clinically relevant ranges of creatinine in a sample.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing,” and “comprising” are interchangeable, and one of the skills in the art is cognizant that these terms are open-ended terms.

As used herein, the term “analyte” or “target” refers to a substance whose chemical constituents are being identified and measured through disclosed systems and arrays. An analyte can include, but is not limited to, all molecules, ions, chemicals, peptides, etc.

As used herein, the term “about” or “approximately” means within an acceptable error range for the particular value, as determined by one of ordinary skill in the art, which will depend, in part, on how the value is measured or determined, (i.e., the limitations of the measurement system). For example, “about” can mean within 3 or more than 3 standard deviations, per the practice of the art.

As used herein, the term “subject” includes any human or nonhuman animal. The term “nonhuman animal” includes, but is not limited to, all vertebrates, (e.g., mammals and non-mammals, such as nonhuman primates, dogs, cats, sheep, horses, cows, chickens, rodents, amphibians, reptiles, etc.). In certain embodiments, the subject is a pediatric patient. In certain embodiments, the subject is an adult patient.

The terms “detection” or “detecting” include any means of detecting, including direct and indirect detection.

As described herein, any concentration range, percentage range, ratio range, or integer range is to be understood to include the value of any integer within the recited range and, when appropriate, fractions thereof (such as one-tenth and one-hundredth of an integer), unless otherwise indicated.

In certain embodiments, the disclosed subject matter provides an oligonucleotide-based receptor (also known as aptamer) that can form a complex with a target molecule/analyte (e.g., creatinine). In non-limiting embodiments, the aptamer can form a stem-loop structure and bind to the target molecule/analyte. In non-limiting embodiments, the formation of such a complex can be characterized by dissociation constants in the range from about 10⁻¹⁰ to about 10⁻¹, from about 10⁻⁹ to about 10⁻¹, from about 10⁻⁸ to about 10⁻¹, from about 10⁻⁷ to about 10⁻¹, from about 10⁻⁶ to about 10⁻¹, from about 10⁻⁶ to about 10⁻¹, from about 10⁻⁵ to about 10⁻¹, from about 10⁻⁴ to about 10⁻¹, from about 10⁻³ to about 10⁻¹, from about 10⁻² to about 10⁻¹, from about 10⁻¹⁰ to about 10⁻², from about 10⁻¹⁰ to about 10⁻³, from about 10⁻¹⁰ to about 10⁻⁴, from about 10⁻¹⁰ to about 10⁻⁵, from about 10⁻¹⁰ to about 10⁻⁶, from about 10⁻¹⁰ to about 10⁻⁷, from about 10⁻¹⁰ to about 10⁻⁸, or from about 10⁻¹⁰ to about 10⁻⁹. In non-limiting embodiments, the dissociation constants of the disclosed aptamer can be between about 10⁻⁹ and about 10⁻⁶ M or in the range between about 10⁻⁶ and about 10⁻³ M.

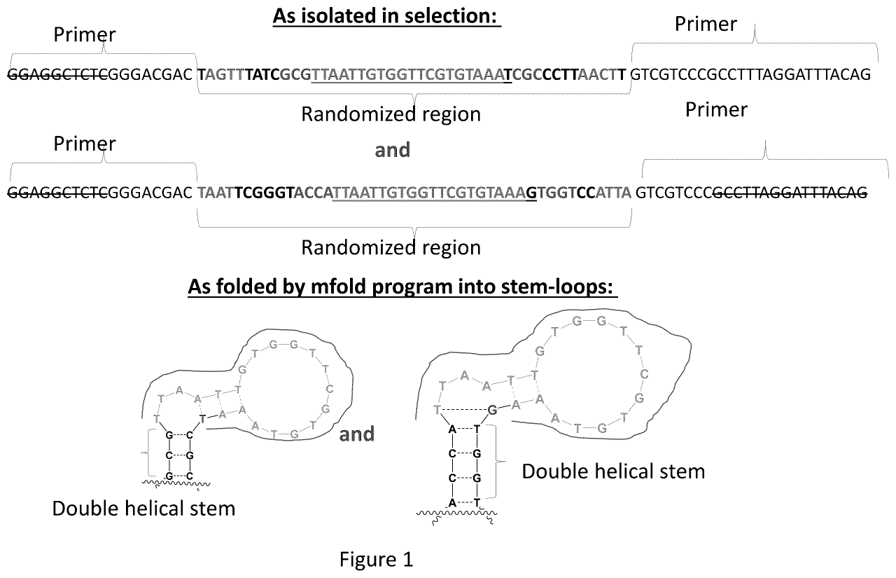

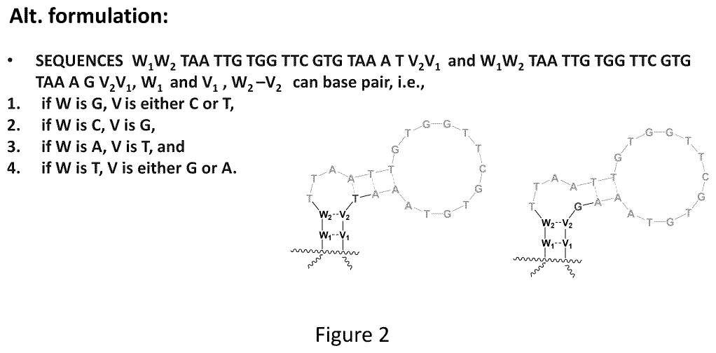

In certain embodiments, the disclosed aptamer can be presented as a single-stranded DNA that can include an oligonucleotide sequence incorporating TAATTGTGGTTCGTGTAAA (SEQ ID NO: 1). The single-stranded DNA can be a part of its larger structure (FIGS. 1 and 2). FIG. 1 shows two example sequences that can be within larger sequences isolated in selections. The example sequences TAA TTG TGG TTC GTG TAA A T (SEQ ID NO: 13) and TAA TTG TGG TTC GTG TAA A G (SEQ ID NO: 14) can connect other sequences that can base-pair to form the larger structure and/or a double helical stem. For example, the structure with the disclosed sequences can be folded into a stem-loop structure (FIG. 1). FIG. 2 shows example sequences that are flanked by sequences that can form base pairs. Examples of non-limiting embodiments of sequences within the disclosed aptamer can be CGTTAATTGTGGTTCGTGTAAATCG (SEQ ID NO: 15), or CATTAATTGTGGTTCGTGTAA AGTG (SEQ ID NO: 16). In non-limiting embodiments, the sequences can include W₁W₂TAATTGTGGTTCGTGTAAATV₂V₁ (SEQ ID NO: 6) or W₁W₂TAATTGTGG TTCGTGTAAAGV₂V₁ (SEQ ID NO: 7). In non-limiting embodiments, W₁ and V₁ can form a base pair, and W₂ and V₂ can form a base pair. For example, if W₁ or W₂ is G, V₁ or V₂ can be either C or T. If W₁ or W₂ is C, V₁ or V₂ can be G. If W₁ or W₂ is A, V₁ or V₂ can be T. If W₁ or W₂ is T, V₁ or V₂ is either G or A.

In certain embodiments, the disclosed aptamer can include sequences of fifteen, sixteen, seventeen, eighteen, nineteen, or twenty bases identical to at least about 60%, at least about 65%, at least about 70%, at least about 75%, or at least about 80% to TAATTGTGGTTCGTGTAAA (SEQ ID NO: 1). The disclosed sequence can include certain modifications (e.g., substitution, insertion, deletion, and/or inversion) as long as it is identical at least about 60%, about 65%, about 70%, about 75%, or about 80%.

In certain embodiments, the disclosed aptamer can include a single-stranded DNA strand and/or an oligonucleotide sequence with at least one modified base. For example, the modified base can include RNA, modified RNA, modified DNA, PNA, or combinations thereof.

In certain embodiments, the disclosed aptamer can include a single-stranded DNA strand and/or an oligonucleotide sequence with modifications in the phosphodiester backbone. For example, these modifications can include atom substitutions, neutralization of negative charges, or introduction of positive charges.

In certain embodiments, the disclosed sequences can be immobilized to a substrate (e.g., metal substrate) and used for sensing a target molecule/analyte. In non-limiting embodiments, upon binding to the target analyte, the disclosed aptamers can reorient (change conformation) and affect the behavior of charge carriers in a substrate (e.g., semiconductor), resulting in a change in the conductance. For example, the disclosed sequences can be labeled for electrochemistry and attached to an electrode (e.g., gold electrode). Upon binding to creatinine, the aptamer structure can be modified to approach to the electrode so the conductance can be altered. In non-limiting embodiments, the aptamer can be configured to modify its structure to move away from a substrate upon binding to a target molecule/analyte.

In certain embodiments, the disclosed aptamer can be synthesized to include functional groups that otherwise do not exist in natural nucleic acids. In non-limiting examples, the groups can include thiols (—SH), products of their reactions (e.g., thioethers, amino (—NH2) and/or carboxyl (—COOH) groups and products of their reactions (amides), aldehydes and/or products of their reactions (imines, hydrozones, hydrazides, or amines), biotin and/or its analogs, and/or any of the functional groups that can be used in click chemistry (e.g., alkynes, azides, strained alkenes, tetrazines) or products of their reactions (e.g., triazoles)).

In certain embodiments, the disclosed aptamer, in its sensor form, can respond to lower concentrations of creatinine than of non-creatinine species, examples of which are creatine, lactate, and pyruvate.

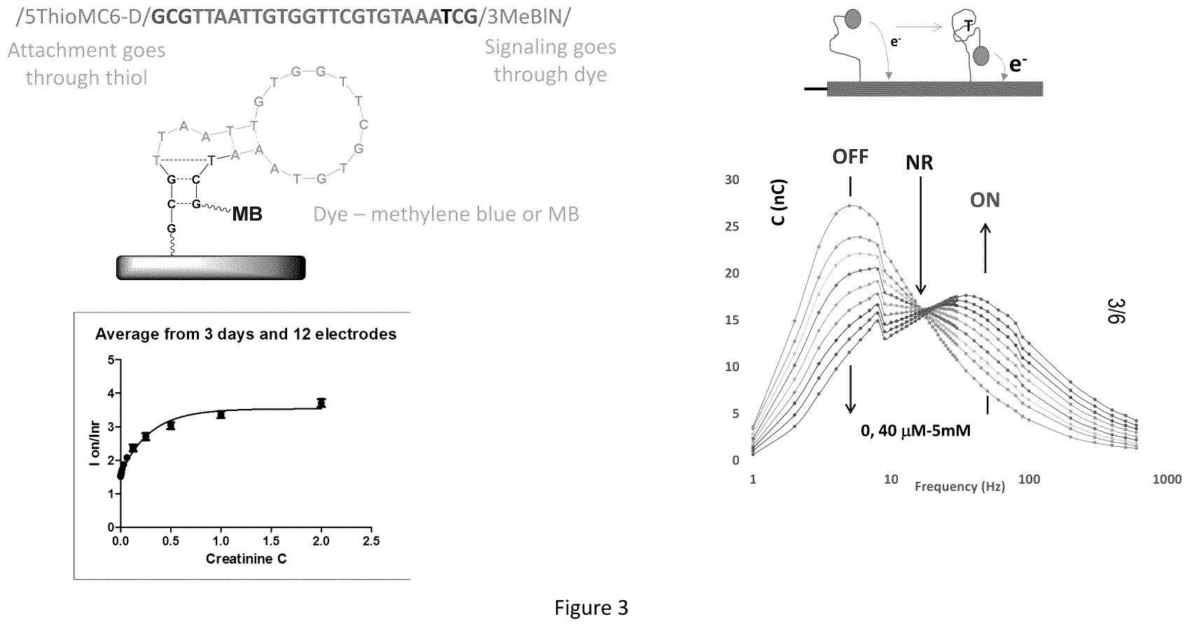

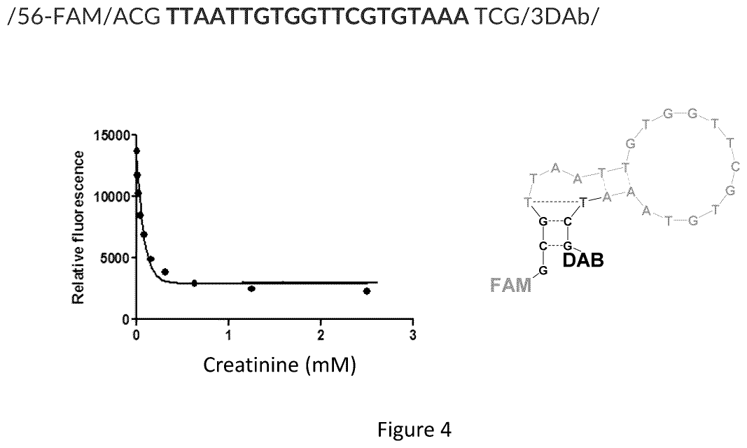

In certain embodiments, the disclosed aptamer can be part of the larger sequence that can be folded into a stem-loop, with sequence TAATTGTGGTTCGTGTAAA (SEQ ID NO: 1) being then part of a sequence that links at least one stem. In some embodiments, such a stem-loop can be unstable in the absence of creatinine, and its formation can be induced by the presence of creatinine (FIGS. 3 and 4).

FIG. 3 shows example sequences that can be attached to solid surfaces (e.g., metal, gold, etc.). In certain embodiments, the disclosed subject matter provides an electronic, electrical or optical device for measuring creatinine based on interactions of an oligonucleotide-based receptor, and this receptor can include the disclosed sequence (FIG. 3). The sensor can report the presence or absence of analytes through a change in fluorescence or a change in an electrochemical readout. In non-limiting embodiments, the sensor can include a combination of a field-effect transistor and the disclosed sequence, a gold surface and the disclosed sequence, a fiber-optical cable and the disclosed aptamer, and/or a quartz surface and the disclosed sequence. In non-limiting embodiments, the disclosed sequence can be a part of a larger sequence that can have more than one conformation, with ligand binding impacting the equilibrium between the conformations. For example, the distance between an internal base within the disclosed sequences and the 5′ end of the sequence can increase or decrease. In some embodiments, the interaction of creatinine and disclosed sequence within a larger sequence can change the extent of binding to dye, resulting in a measurable impact on optical or electrochemical properties.

In certain embodiments, the disclosed sequence can be attached to larger objects without the ability to freely diffuse through a solution. In non-limiting embodiments, the disclosed subject matter provides a freely diffusing molecular device with a molecular weight between about 1,000 D and about 1,000,000 D. In non-limiting embodiments, for measuring creatinine based on interactions of an oligonucleotide-based receptor and this receptor, the disclosed diffusing molecular device can include the disclosed sequence. The molecular device can report the presence through changes in the optical or electrical properties of a solution.

In non-limiting embodiments, the disclosed devices, which can be either freely diffusing or attached to larger objects, can include a fluorophore, a quencher, an enzyme, or a redox dye. A fluorophore, a quencher, a redox dye, and/or a chemical modifier can be connected at 5′ end, 3′ end, or both ends of the disclosed sequences. FIG. 4 shows an example sequence that can send creatinine in a solution (e.g., urine, blood, serum, etc.) when modified with fluorophores and/or quenchers. FIG. 4 shows an example sequence that can send creatinine in a solution (e.g., urine, blood, serum, etc.) when modified with fluorophores and/or quenchers. As shown in the graph in FIG. 4, the disclosed sequence with fluorophores and quenchers (e.g., FAM and DAB) can decrease the level of relative fluorescence as the level of creatinine increases. In non-limiting embodiments, the fluorophore can be any available fluorophore (e.g., Alexa Fluor, Cy2, Cy3, Cy5, Cy7, coumarin, TRITC, FITC, Qdot, DAPI, SYTOX Green, SYTO 9, TO-Pro-3, eFluor, PE-efluor, PE-cyanine7, Pacific Blue, Pacific Orange, Texas Red, etc.). In non-limiting embodiments, the redox dye can be any available redox dye. For example, the redox dye can be methylene blue and its analog, malachite green, or its analog, anthracene, Neutral red, Safranin T, Phenosafranin, Indigomono sulfonic acid, Indigo carmine, Indigotrisulfonic acid, Indigotetrasulfonic acid, Thionine, Sodium o-Cresol indophenol, Sodium 2,6-Dibromophenol-indophenol, Viologen, Diphenylamine, Diphenylbenzidine, Sodium diphenylamine sulfonate, o-Dianisidine, 5,6-Dimethylphenanthroline, 2,2-Bipyridine, N-Ethoxychrysoidine, 1,10-Phenanthroline iron (II) sulfate complex, N-Phenylanthranilic acid, Nitrophenanthroline, 2,2′-bipyridine, or analogs thereof. In certain embodiments, the quencher can be any available quencher (e.g., dabcyl, Iowa Black, TAMRA, BHQ, BBQ, Atto, MGB, or Dab). In certain non-limiting embodiments, the enzyme can be any enzyme that can be used in electrochemistry (e.g., glucose oxidase, glucose hexokinase, etc.).

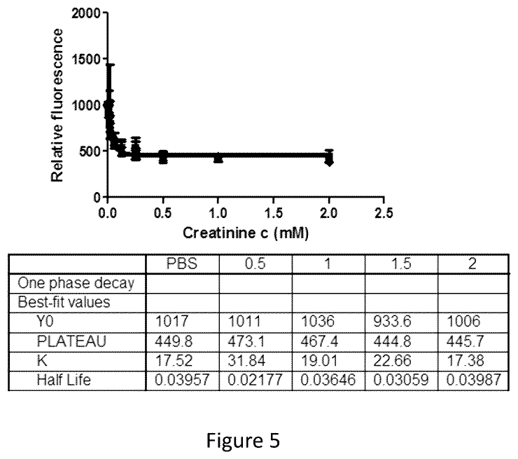

In certain embodiments, the disclosed aptamer can bind to creatinine despite physiological variations in concentrations of ions, such as magnesium, potassium, calcium, sodium, or combinations thereof. For example, Mg(II) ion can vary from 0.25-1.75×10⁻³ M, and the extent of the complex formation with the aptamer encompassing the disclosed sequence can minimally change (FIG. 5).

The disclosed aptamers and systems can be unresponsive to Mg(II), potassium, and sodium concentrations in their physiological ranges, which can make them uniquely suitable for direct measurements in certain samples even without further buffering (FIG. 5). The sample can include blood, serum, effluent, or combinations thereof. The disclosed aptamers and system can also provide sensitivity to creatinine in the concentration range between about 10⁻⁶ to about 10⁻³ M, which can make them also suitable for direct use in serum, whole blood or interstitial, lymphatic, dialysis effluent fluids, saliva, sweat or tears. In non-limiting embodiments, the disclosed sequences can have low sensitivity to physiological level of non-creatinine analytes or molecules (e.g., magnesium, potassium, calcium, or sodium), allowing more accurate and direct measurement of creatinine in physiological samples (e.g., blood, serum, urine, etc.) without additional buffer.

In certain embodiments, the disclosed aptamers and systems can detect creatinine in diluted or undiluted samples. For example, by mixing the disclosed aptamers with samples and reading fluorescent or electrical or colorimetric signal that is proportional to the concentration of creatinine, the disclosed system can detect creatinine in the diluted samples. The sample can include blood, serum, effluent, or combinations thereof. In non-limiting embodiments, the diluted samples can be a sample diluted up to about 1%, 10%, 20%, 30%, 40%, 50%, 60%, 70%, 90%, 100%, 200%, 300%, 400%, 500%, 600%, 700%, 800%, 900%, or 1000% with a solution.

The disclosed subject matter provides methods for diagnosing a kidney disease of a subject. An example method can include contacting at least a portion of a sample of a subject with the disclosed aptamer, detecting a level of creatinine of the sample using the aptamer, and providing the subject with appropriate treatments based on the detected level of creatinine in the sample. As creatinine is a waste product that comes from the metabolism of muscles of the body, creatinine levels in the sample can be used to assess the status of the kidneys. As kidney disease progresses, the level of creatinine in the sample can rise. In non-limiting embodiments, appropriate treatments for kidney injuries can be provided to a subject/patient depending on the level of detected creatinine level. For example, the level of kidney treatment can be enhanced with the patient with increased creatinine level detected by the disclosed system. The level of the kidney treatment can be lowered with the patient with decreased creatinine level detected by the disclosed system.

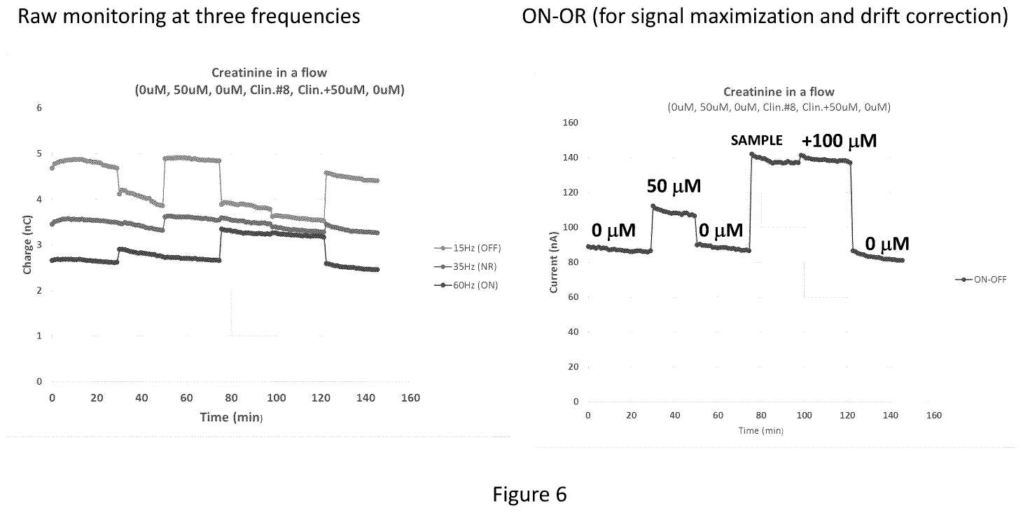

In certain embodiments, as shown in FIG. 6, the disclosed sequences can be part of larger structures for sensing creatinine at predetermined frequencies (e.g., once every hour, once every ten minutes, once every minute, once every second, or any predetermined period in between). FIG. 6 provides graphs showing continuous monitoring of the disclosed subject matter using square wave voltammetry in buffers and clinical samples. In non-limiting embodiments, the rate of change of creatinine can be used to assess dialysis adequacy, a dose of dialysis delivered, progression, removal of other materials by dialysis, or subject's health in general (e.g., in circumstances of fluctuating or rapid production of creatinine, acute kidney injury, recovering acute kidney injury, rhabdomyolysis, etc.). In non-limiting examples, the rate of creatinine change in the blood, plasma, serum, or effluent fluid during any form of dialysis or hemofiltration can be used to adjust parameters for dialysis or hemofiltration or to adjust the dosing of other drugs or identify circumstances where creatinine concentrations can be fluctuating.

In certain embodiments, the disclosed sequences can be modified to improve their stability and/or affinity. For example, certain residues of the disclosed sequences can be added, deleted, or replaced to improve their stability and/or affinity. In non-limiting embodiments, modifications of the length of the stem and/or loop can adjust the nature of the binding mechanism, thermodynamics, affinity, and thermal stability of the disclosed aptamers. For example, at least one, two, three, four, five, six, seven, eight, nine, ten, fifteen, or twenty base pairs of the stem and/or loop of the disclosed aptamers can be added, deleted, or replaced for improving thermal stability and/or affinity to the target molecules. In non-limiting embodiments, the lengthened sequence of the stem can result in the increased binding affinity. The shortened sequence of the stem can result in the increased thermal stability. The lengthened sequence of the stem can result in tighter binding to the target molecules. The improved aptamers can have higher melt temperatures when they bind to a ligand. The improved aptamers can include any variants that are apparent to those skilled in the art in view of the teachings herein

In certain embodiments, the affinity and thermal stability of the disclosed aptameric receptors and sensors can be derived from the modification of the disclosed sequences/aptamers. For example, the affinity and thermal stability of the disclosed aptameric receptors and sensors can be further adjusted by the length and composition of double helical stems and single point mutations. In non-limiting embodiments, the stability and/or affinity of the disclosed sequences can be verified through any relevant techniques. For example, the stability and/or affinity of the disclosed sequences can be verified through isothermal titration calorimetry or NMR spectroscopy.

The description herein merely illustrates the principles of the disclosed subject matter. Various modifications and alterations to the described embodiments will be apparent to those skilled in the art in view of the teachings herein. Further, it should be noted that the language used herein has been selected for readability rather than to delineate or limit the disclosed subject matter. Accordingly, the disclosure herein is intended to be illustrative, but not limiting, of the scope of the disclosed subject matter.

What is claimed is:

1. An aptamer for detecting a target molecule/analyte in a sample comprising a single-stranded deoxyribonucleic acid (DNA) strand that includes an oligonucleotide sequence with bases identical at least about 60% to TAATTGTGGTTCGTGTAAA (SEQ ID NO: 1), wherein the aptamer is configured to bind the target molecule/analyte with a dissociation constant between about 10⁻⁹ and about 10⁻³ M.

2. The aptamer of claim 1, wherein the target molecule/analyte is creatinine.

3. The aptamer of claim 1, wherein the single-stranded DNA strand comprises at least about fifteen bases.

4. The aptamer of claim 1, wherein the aptamer comprises at least one modification.

5. The aptamer of claim 4, wherein the at least one modification is selected from the group consisting of atom substitution, neutralization of negative charges, introduction of positive charges, and combinations thereof.

6. The aptamer of claim 4, wherein the at least one modification is a modified base, wherein the modified base is selected from the group consisting of a ribonucleic acid (RNA), a modified RNA, a modified DNA, a peptide nucleic acid (PNA), and combinations thereof.

7. The aptamer of claim 1, wherein the sample comprises blood, serum, effluent, saliva, sweat, tears, or combinations thereof.

8. The aptamer of claim 1, wherein the aptamer comprises a functional group, wherein the functional group comprises thiols, phosphothiols, carboxyl, amines, carbonyls, aldehydes, alkynes, azides, alkenes, strained alkenes, tetrazines, and/or products thereof.

9. The aptamer of claim 1, wherein the aptamer is a stem-loop aptamer that includes a capture region and a stem region, wherein the stem region is configured to be positioned to transform a second conformation into a stem-loop structure of the aptamer, or stem-loop structure into a second conformation when the oligonucleotide sequence binds to the target molecule/analyte.

10. The aptamer of claim 1, wherein the aptamer is modified or configured to be immobilized to a substrate for sensing the target molecule/analyte.

11. The aptamer of claim 9, wherein the stem-loop structure is modified to move away from the substrate upon binding to the target molecule/analyte.

12. The aptamer of claim 9, wherein the stem-loop structure is modified to approach to the substrate upon binding to the target molecule/analyte.

13. The aptamer of claim 1, wherein the aptamer is configured to be incorporated into a sensor device, wherein the sensor device comprises a field-effect transistor and the aptamer.

14. The aptamer of claim 1, wherein the aptamer is configured to be incorporated into a sensor device, wherein the sensor device comprises a gold substrate and the aptamer.

15. The aptamer of claim 1, wherein the aptamer is configured to be incorporated into a sensor device, wherein the sensor device comprises a fiber-optical cable and the aptamer.

16. The aptamer of claim 1, wherein the aptamer is configured to be incorporated into a sensor device, wherein the sensor device comprises a quartz surface and the aptamer.

17. The aptamer of claim 1, wherein the aptamer is configured to the target molecule/analyte without being freely diffused.

18. The aptamer of claim 13, wherein the sensor device comprises a freely diffusing molecular with a molecular weight between about 1,000 D and about 1,000,000 D.

19. The aptamer of claim 1, wherein the aptamer is attached to a fluorophore, a quencher, an enzyme, a redox dye, or combinations thereof.

20. The aptamer of claim 1, wherein the sample is a diluted sample, wherein the diluted sample is a sample diluted up to about 1%, 10%, 20%, 30%, 40%, 50%, 60%, 70%, 90%, 100%, 200%, 300%, 400%, 500%, 600%, 700%, 800%, 900%, or 1000% with a solution.

21. The aptamer of claim 1, wherein the sample is a non-diluted sample.

22. A method for detecting a target molecule/analyte in a sample, comprising:
contacting at least a portion of the sample with effective amounts of an aptamer, wherein the aptamer comprises a single-stranded deoxyribonucleic acid (DNA) strand that includes an oligonucleotide sequence with bases identical at least about 60% to TAATTGTGGTTCGTGTAAA (SEQ ID NO: 1), wherein the aptamer is configured to bind the target molecule/analyte with a dissociation constant between about 10⁻⁹ and about 10⁻³ M,
detecting a change after the contacting.

23. The method of claim 22, wherein the change comprises a change of conductance, fluorescence, and/or any electrochemical readouts.

24. The method of claim 22, wherein the target molecule/analyte is creatinine.

25. The method of claim 22, wherein the sample comprises blood, plasma, serum, effluent, saliva, sweat, tears, or combinations thereof.

26. The method of claim 22, wherein the aptamer is a stem-loop aptamer that includes a capture region and a stem region, wherein the stem region is configured to be positioned to transform a stem-loop structure of the aptamer to a second conformation when the oligonucleotide sequence binds to the target molecule/analyte.

27. The method of claim 22, wherein the aptamer is configured to be immobilized to a substrate for sensing the target molecule/analyte.

28. The method of claim 27, wherein the substrate comprises gold and/or quartz.

29. The method of claim 26, wherein the stem-loop structure is modified to move away from the substrate upon binding to the target molecule/analyte so the conductance of the substrate changes.

30. The method of claim 26, wherein the stem-loop structure is modified to approach to the substrate upon binding to the target molecule/analyte so the conductance of the substrate changes.

31. The method of claim 22, wherein the aptamer is attached to a fluorophore, a quencher, an enzyme, a redox dye, or combinations thereof.

32. The method of claim 22, wherein the sample is a diluted sample, wherein the sample is a diluted sample, wherein the diluted sample is a sample diluted up to about 1%, 10%, 20%, 30%, 40%, 50%, 60%, 70%, 90%, 100%, 200%, 300%, 400%, 500%, 600%, 700%, 800%, 900%, or 1000% with a solution.

33. The method of claim 22, wherein the sample is a non-diluted sample.

34. The method of claim 22, further comprising
diagnosing a kidney disease based on the detected level of creatinine in the sample, and
providing a subject with a treatment based on the detected level of creatinine in the sample.

35. The method of claim 34, wherein a level of the treatment is enhanced with an increased creatinine level detected by the aptamer.

36. The method of claim 34, wherein a level of the treatment is lowered with a decreased creatinine level detected by the aptamer.

37. The method of claim 22, further comprising sensing the target molecule/analyte at a predetermined frequency.

38. The method of claim 37, the predetermined frequency comprises once every hour, once every ten minutes, once every minute, once every second, or any predetermined period in between.


SEQUENCE LISTING (16 sequences)

SEQ ID NO: 1 - DNA, 19 bp, synthetic construct
  misc_feature 1..19: note = Synthetic
  source 1..19: mol_type = other DNA
taattgtggttcgtgtaaa

SEQ ID NO: 2 - DNA, 88 bp, synthetic construct
  misc_feature 1..88: note = Synthetic
  source 1..88: mol_type = other DNA
ggaggctctcgggacgactagtttatcgcgttaattgtggttcgtgtaaatcgcccttaacttgtcgtcccgcctttaggatttacag

SEQ ID NO: 3 - DNA, 87 bp, synthetic construct
  misc_feature 1..87: note = Synthetic
  source 1..87: mol_type = other DNA
ggaggctctcgggacgactaattcgggtaccattaattgtggttcgtgtaaagtggtccattagtcgtcccgccttaggatttacag

SEQ ID NO: 4 - DNA, 27 bp, synthetic construct
  misc_feature 1..27: note = Synthetic
  source 1..27: mol_type = other DNA
gcgttaattgtggttcgtgtaaatcgc

SEQ ID NO: 5 - DNA, 29 bp, synthetic construct
  misc_feature 1..29: note = Synthetic
  source 1..29: mol_type = other DNA
accattaattgtggttcgtgtaaagtggt

SEQ ID NO: 6 - DNA, 24 bp, synthetic construct
  misc_feature 1..24: note = Synthetic
  misc_difference 1..2: note = if n is G, then n at positions 23 and 24 is C or T
  misc_difference 1..2: note = if n is C, then n at positions 23 and 24 is G
  misc_difference 1..2: note = if n is A, then n at positions 23 and 24 is T
  misc_difference 1..2: note = if n is T, then n at positions 23 and 24 is G or A
  misc_difference 23..24: note = if n at positions 1 and 2 is G, then n at positions 23 and 24 is C or T
  misc_difference 23..24: note = if n at positions 1 and 2 is C, then n at positions 23 and 24 is G
  misc_difference 23..24: note = if n at positions 1 and 2 is A, then n at positions 23 and 24 is T
  misc_difference 23..24: note = if n at positions 1 and 2 is T, then n at positions 23 and 24 is G or A
  source 1..24: mol_type = other DNA
nntaattgtggttcgtgtaaatnn

SEQ ID NO: 7 - DNA, 24 bp, synthetic construct
  misc_feature 1..24: note = Synthetic
  misc_difference 1..2: note = if n is G, then n at positions 23 and 24 is C or T
  misc_difference 1..2: note = if n is C, then n at positions 23 and 24 is G
  misc_difference 1..2: note = if n is A, then n at positions 23 and 24 is T
  misc_difference 1..2: note = if n is T, then n at positions 23 and 24 is G or A
  misc_difference 23..24: note = if n at positions 1 and 2 is G, then n at positions 23 and 24 is C or T
  misc_difference 23..24: note = if n at positions 1 and 2 is C, then n at positions 23 and 24 is G
  misc_difference 23..24: note = if n at positions 1 and 2 is A, then n at positions 23 and 24 is T
  misc_difference 23..24: note = if n at positions 1 and 2 is T, then n at positions 23 and 24 is G or A
  source 1..24: mol_type = other DNA
nntaattgtggttcgtgtaaagnn

SEQ ID NO: 8 - DNA, 25 bp, synthetic construct
  misc_feature 1..25: note = Synthetic
  misc_difference 1..2: note = if n is G, then n at positions 24 and 25 is C or T
  misc_difference 1..2: note = if n is C, then n at positions 24 and 25 is G
  misc_difference 1..2: note = if n is A, then n at positions 24 and 25 is T
  misc_difference 1..2: note = if n is T, then n at positions 24 and 25 is G or A
  misc_difference 24..25: note = if n at positions 1 and 2 is G, then n at positions 24 and 25 is C or T
  misc_difference 24..25: note = if n at positions 1 and 2 is C, then n at positions 24 and 25 is G
  misc_difference 24..25: note = if n at positions 1 and 2 is A, then n at positions 24 and 25 is T
  misc_difference 24..25: note = if n at positions 1 and 2 is T, then n at positions 24 and 25 is G or A
  source 1..25: mol_type = other DNA
nnttaattgtggttcgtgtaaatnn

SEQ ID NO: 9 - DNA, 25 bp, synthetic construct
  misc_feature 1..25: note = Synthetic
  misc_difference 1..2: note = if n is G, then n at positions 24 and 25 is C or T
  misc_difference 1..2: note = if n is C, then n at positions 24 and 25 is G
  misc_difference 1..2: note = if n is A, then n at positions 24 and 25 is T
  misc_difference 1..2: note = if n is T, then n at positions 24 and 25 is G or A
  misc_difference 24..25: note = if n at positions 1 and 2 is G, then n at positions 24 and 25 is C or T
  misc_difference 24..25: note = if n at positions 1 and 2 is C, then n at positions 24 and 25 is G
  misc_difference 24..25: note = if n at positions 1 and 2 is A, then n at positions 24 and 25 is T
  misc_difference 24..25: note = if n at positions 1 and 2 is T, then n at positions 24 and 25 is G or A
  source 1..25: mol_type = other DNA
nnttaattgtggttcgtgtaaagnn

SEQ ID NO: 10 - DNA, 26 bp, synthetic construct
  misc_feature 1..26: note = Synthetic
  modified_base 1: mod_base = OTHER; note = 5'Thiol Modifier C6 S--S
  modified_base 26: mod_base = OTHER; note = 3' methylene blue modifier
  source 1..26: mol_type = other DNA
gcgttaattgtggttcgtgtaaatcg

SEQ ID NO: 11 - DNA, 26 bp, synthetic construct
  misc_feature 1..26: note = Synthetic
  modified_base 1: mod_base = OTHER; note = 5'6-Carboxyfluorescein
  modified_base 26: mod_base = OTHER; note = 3' 4-(dimethylaminophenylazo)benzoic-4-carboxylic acid
  source 1..26: mol_type = other DNA
acgttaattgtggttcgtgtaaatcg

SEQ ID NO: 12 - DNA, 26 bp, synthetic construct
  misc_feature 1..26: note = Synthetic
  modified_base 1: mod_base = OTHER; note = Carboxyfluorescein
  modified_base 26: mod_base = OTHER; note = 4-(dimethylaminophenylazo)benzoic-4-carboxylic acid
  source 1..26: mol_type = other DNA
gcgttaattgtggttcgtgtaaatcg

SEQ ID NO: 13 - DNA, 20 bp, synthetic construct
  misc_feature 1..20: note = Synthetic
  source 1..20: mol_type = other DNA
taattgtggttcgtgtaaat

SEQ ID NO: 14 - DNA, 20 bp, synthetic construct
  misc_feature 1..20: note = Synthetic
  source 1..20: mol_type = other DNA
taattgtggttcgtgtaaag

SEQ ID NO: 15 - DNA, 25 bp, synthetic construct
  misc_feature 1..25: note = Synthetic
  source 1..25: mol_type = other DNA
cgttaattgtggttcgtgtaaatcg

SEQ ID NO: 16 - DNA, 25 bp, synthetic construct
  misc_feature 1..25: note = Synthetic
  source 1..25: mol_type = other DNA
cattaattgtggttcgtgtaaagtg